(19) United States
(12) Patent Application Publication
(10) Pub. No.: US 2011/0282451 A1
(43) Pub. Date: Nov. 17, 2011

(54) MEDICAL COMPONENT

(76) Inventors: Sune Lund Sporring, Naestved (DK); Ole Olsen, Charlottenlund (DK); Tom Løgstrup Andersen, Roskilde (DK); Povl Brøndsted, Koge (DK); Mischa Nelis, Simpelveld (NL); Roelof Marissen, Born (NL)

(21) Appl. No.: 12/922,953

(22) PCT Filed: Mar. 17, 2009
(86) PCT No.: PCT/EP2009/001953

§ 371 Date: Jan. 6, 2011

(30) Foreign Application Priority Data
Mar. 17, 2008 (DK) PA200800411

(51) Int. Cl. A61F 2/08; B23P 17/04; B32B 7/02

(52) U.S. Cl. 623/14.12; 428/212; 29/428

(57) ABSTRACT

A biocompatible medical component comprising•a hybrid fabric comprising i. at least one type of second fibre, and ii. at least one type of first fibre, and•a structural component, wherein the Young's modulus of said second fibre is at least 125% of the Young's modulus of the first fiber.

### FIELD OF INVENTION

The present invention relates to medical components such as implants or parts of implants. Particularly, the invention relates to medical components, which are exposed to wear and/or tear during use.

### BACKGROUND OF INVENTION

Medical devices such as implants may be subjected to tear and wear due to movement of an individual into who an implant is transplanted. Particles may be torn from the implant as the implant is being worn. Also the particles from the implant may give risk of an inflammatory reaction within the body of the individual. In some cases the implant has to be removed from the individual as the inflammatory reaction may destroy the tissue and thus give rise to osteolysis of the tissue to where the implant is connected.

It is thus important to develop medical devices which have improved properties in respect of wear and tear, which is not destroyed when in use as an implant in an individual, and which reduces the risk of obtaining inflammatory reactions and osteolysis.

WO 2005/065911 (DSM IP ASSETS B.V) describes a process for manufacturing a prosthetic joint with at least one loaded surface which consists at least partially of polyethylene, comprising compressing one or more layers of a woven fabric of drawn gel-spun polyethylene fibres into the desired shape in a hollow mould part using a plug at a pressure of at least 0.05 MPa and at a temperature of between 120 and 165° C. and below the crystalline melting point of the polyethylene at the prevailing temperature and pressure, without a matrix material being present, and at least the woven fabric in a layer situated on a loaded surface comprising at least 90 wt % of fibres with a titer of at most 1000 denier, and to a prosthetic joint with a crease free surface.

### SUMMARY OF INVENTION

A first aspect of the invention relates to a biocompatible medical component comprising a hybrid fabric comprising at least one type of second fibre, and at least one type of first fibre, and the medical component further comprises a structural component. The first fibre and the second fibre vary at least in that the Young's modulus of said second fibre is at least 125% of the Young's modulus of the first fiber. The hybrid fabric may have a 3D structure with second fibre at least at the side which is to be subjected to wear when positioned in an individual, and first fibre at least at the side which is the anchoring layer i.e. a part of the hybrid fabric which is to be connected to the structural component.

The chemical composition of the first fibre and the second fibre may be the same and vary solely in molecular weight or degree of crystallization or orientation (such as both the type of first fibre and type of the second fibre being polyethylene fibre, such as a LDPE and a UHMWPE), but in most cases, the chemical composition of the first fibre and the second fibre is dissimilar. The hybrid fabric is produced of fibre of material having different properties e.g. metal, ceramic, glass and/or polymer. Especially polymer is suited. Polyolefines such as PE is preferred as second fibre and first fibre. Most preferred is that at least one of the second and first fibre comprises or consist of ultra high molecular weight polyethylene, UHMWPE. Typically, UHMWPE fibre is considered a second fibre when being high crystalline fibre, e.g. highly oriented fibre, and first fibre when being semi-crystalline fibre or even amorphous. However, if UHMWPE high crystalline fibre is combined with a fibre having an even higher Young's modulus, then UHMWPE high crystalline fibre is considered a first fiber, and if UHMWPE (semi-crystalline) fibre is combined with a fibre having an even lower Young's modulus, then the UHMWPE (semi-crystalline) fibre is considered a second fibre.

Especially the hybrid fabric can have second fibre at the side not connected to the medical device or the structural component and first fibre at the side connected to the medical device or the structural component. The hybrid fabric may be interwoven such that the amount of strong fibre gradually decreases from the outside of the fabric towards the side adhered to the implant, whereas the amount of first fibre gradually increases from the outside of the fabric towards the side adhered to the implant. The medical devices or structural component coated with the hybrid fabric may be any medical devices or structural component, but especially implants to be placed into a joint of an individual may be improved by an at least partial cover of the hybrid fabric and especially when covered in areas directly subjected to tear and/or wear when located in an individual due to movements of the individual. All patent and non-patent references cited in the application, or in the present application, are also hereby incorporated by reference in their entirety.

An adhesive or matrix material may be positioned between the hybrid fabric and the structural component before performing a process of connecting the hybrid fabric to the structural component. Hereby the structural component is coated with the hybrid fabric. In another embodiment of the invention, the hybrid fabric and the structural component is connected without the use of adhesion or matrix material. In this embodiment, the connection may for example be established by partial melting of the soft component or by physical means, such as intermingling of fibers or mechanical means microscopically (filament or fibre level) and/or macroscopically (fabric level). Another aspect relates to a method for the production of a medical component, the method comprising providing a hybrid fabric, and providing a structural component, connecting said hybrid fabric to said structural component, and hereby obtaining a medical component.

The connection process may be a compression process, but also a structural component may be injection moulded onto a hybrid fabric, or a partial fusion of the hybrid fabric or the structural component.

Due to the hybrid fabric described herein, the medical devices coated with such hybrid fabric give rise to lower amount of particles when implanted into an individual and thus reduces the risk of inflammatory reactions and osteolysis. The implant is worn more slowly due to second fibre and can therefore last longer, which reduces the risk of re-operation of individuals to replace an implanted implant.

### DESCRIPTION OF DRAWINGS

FIG. 1 shows different typical 2D weave structures which may be used in respect of the preparation of a 3D hybrid fabric as described herein.

FIG. 2 shows a 3D weave structure with multi-layer interlaced.

FIG. 3 illustrates a hybrid fabric (composite fabric)

FIG. 4 illustrates an example of the fibre filaments of a hybrid fibre by a cross section of the hybrid yarn.

FIG. 5 illustrates a spacer fabric as a hybrid or composite fabric. Between an upper fabric layer and a lower fabric layer fibres of filaments are keeping the integrity of the construction.

FIG. 6 illustrates the structure of a medical component.

FIG. 7 illustrates an implant for total hip replacement with an acetabular shell, a liner, a femoral head, a neck and a stem.

FIG. 8 illustrates two examples of a femoral head located in a liner or acetabular shell.

### DETAILED DESCRIPTION OF THE INVENTION

In an aspect the invention relates to a biocompatible medical component comprising

a hybrid fabric comprising

i. at least one type of second fibre, and

ii. at least one type of first fibre, and

a structural component.

In a preferred embodiment, the first fibre and the second fibre vary at least in that the Young's modulus of said second fibre is at least 125% of the Young's modulus of the first fiber.

In a preferred embodiment the structural component and the hybrid fabric is connected to each other such that a first side of the fabric is connected to the structural component. The first side of the hybrid fabric i.e. the side adhered to the structural component comprises a high amount of first fibre, whereas the second side of the hybrid fabric, which will be the outer part of the medical component, comprises a high amount of second fibre. The outer part of the medical component may also be the outer part of a part or section of a medical implant. The second side of the hybrid fabric i.e. the side with a high amount of second fibre is very suitable to be used where two bones, a bone and an implant or where two parts of an implant moves relative to each other.

A hybrid fabric may be considered as a hybrid based on the fabric level, the fiber level and/or the fibre filament level. By hybrid is meant that it contains at least two types of fibres exhibiting different properties, particularly at least one first fibre and at least one second fibre.

At fabric level, the hybrid fabric may for example be interwoven layers of at least one layer of second fibres and at least one layer of first fibre and/or hybrid fibre. Other examples of hybrid fabrics are knitted fabrics, crochet fabric, embroided fabric, 3D fabric, felted fabric tufted fabric, (filament) winded fabric and non-woven fabric.

At the fibre level a hybrid fabric may be prepared from at least one type of second fibre and at least one type of first fibre and/or hybrid fibre. A hybrid fibre may for example be obtained by mixing of second fibre and first fibre, for example by twisting or braiding. Hybrid fibres may also comprise a mixture of first fibre with another type of first fibre or a mixture of second fibre with another type of second fibre. At the fibre filament level a hybrid fabric may be prepared from at least one type of second fibre of second fibre filaments and/or at least one type of hybrid fibre prepared from first fibre filaments and second fibre filaments.

The term “fibre” can also be understood as “thread” or “wire”. A combination of the different types hybrid constructions may be prepared, such that a fabric may be a hybrid fabric based on hybrid at the fabric level and/or hybrid at the fibre level and/or the hybrid at the fibre filament level.

The hybrid fabric as well as the fibre and hybrid fibre are described herein below.

The structural component may be any component or part of a component giving structure to a medical device. Examples of structural components are further described herein.

### Hybrid Fabric (Second Fibre, First Fibre, Hybrid Fibre)

In an embodiment the second fibre of the hybrid fabric can be
a metal fibre selected from the group of metals normally used for implants to be located in an individual like stainless steel, titanium or cobalt/chrome steels and/or
a polymer fibre selected from the group of polyolefins e.g. UHMWPE; aramid or para-aramid (Kevlar), polyketones e.g. PEEK (polyetheretherketone), polyamide e.g. Nylon, polyester, polyvinylchloride and polyacrylonitrile and/or
a carbon fibres or carbon nanotubes, and/or
a ceramic or glass fibre selected from the group of SiC, Si₃N₄, Ca₃(PO₄)₂, SiO₂, CaO, MgO, K₂O, Na₂O, Al₂O₃, silicates, boro silicates, alumino silicates, BC, mineral fibers and bone ingrowth materials, such as PMMA, FiberLive™, tricalciumphosphate, hydroxyapatite covered materials, porous materials, such as tantalum, titanium or steel and/or
a natural fibre selected from the group of silk, spider silk, plant fibre e.g. cellulose (e.g. from cotton and hemp or from other plants) and/or
fibre selected from the group of coated fibre, coextruded fibre and hybrid fibres and/or
any mixture thereof on fabric level, fibre level or filament level.

The polymers to be used are the family of synthetic or natural macromolecules consisting of inorganic, organic polymers and combinations thereof.

Organic polymers may be natural, synthetic, copolymers, or semisynthetic polymers. Natural polymers comprise of the class of compounds known as polysaccharides, polypeptides, and hydrocarbons such as rubber and polyisoprene. Synthetic polymers comprise elastomers such as nylon, polyvinyl resin, polyvinyl chloride, polyvinyl dichloride, polyvinylpyrrolidone, polyethylene, polystyrene, polypropylene, polyurethane, fluorocarbon resins, acrylate resins, polyacrylates, polymethylmethacrylate, linear and cross-linked polyethylene, phenolics, polyesters, polyethers, polypyrolidone, polysulfone, polyterpene resin, polytetrafluoroethylene, polythiadiazole, polyvinylalcohol, polyvinylacetal, polyvinyl oxides, and alkyds. Semisynthetic polymers may be selected from cellulosics such as rayon, methylcellulose, cellulose acetate and modified starches. Polymers may be atactic, stereospecific, stereoregular or stereoblock, linear, cross-linked, block, graft, ladder, high, and/or syndiotactic. The term graft polymer is intended to mean copolymer molecules comprising a main backbone to which side chains are attached. The main chain may be a homopolymer or copolymer and the side chains may contain different inorganic or organic constituents.

Preferred is when polymer fibre is selected from the group of polyolefinic polymers, polyethylene, polypropylene, polyacrylates, polystyrene, polytetrafluorethylene, polyvinylalcohol, polyethylene oxides, polyvinylpyrrolidon, polysilanes, polyurethanes, polyethers, polyamides, polyesters, polyalkyl acrylates, nylon, rubber and/or epoxy resins. It should be understood that the above list of polymers is not exhaustive, and other polymers may also be employed in the present invention. Preferred is polyethylene and polypropylene. Most preferred is polyethylene. Preferably, the polymer materials of the second fiber may be from the group of polyethylenes or the group of polypropylenes such as polyethylene (PE), polypropylene (PP), high molecular weight polypropylene (HMWPP), high molecular weight polyethylene (HMWPE), ultra high molecular weight polyethylene (UHMWPE) and ultra high molecular weight polypropylene (UHMWPP), high density polyethylene (HDPE), low density polyethylene (LDPE), high density polypropylene (HDPP) and low density polypropylene (LDPP), ultra high density polyethylene (UHDPE), ultra high density polypropylene (UHDPP), cross-linked polyethylene, non-cross-linked polyethylene, cross-linked polypropylene and/or non-cross-linked polypropylene. In this embodiment of the present invention, any combination of polymers listed above, or their equivalents, may be used. In another embodiment the second fibre is high crystalline fibre, such as melt spun or gel spun polyethylene. Especially gel-spun UHMWPE high crystalline fibre is suitable due to a combination of high strength, high modulus and biocompatibility. In another preferred embodiment the hybrid fabric has a high tensile strength and a high wear resistance. The degree of tensile strength is determined by the polymer utilised to produce the fibre and the thickness of the fibre. The tensile strength of the strand or fibre at least of the second fibre and/or of the hybrid fibre in a hybrid fabric is preferably above 1.0 GPa, such as above 1.2 GPa, preferable above 1.4 GPa, more preferable above 1.6 GPa, further preferable above 1.8 GPa, yet further preferable above 1.9 GPa, more preferable above 2.0 GPa, and most preferable above 3.0 GPa.

In another embodiment the tensile strength of the strand or fibre at least of the first fibre and/or of the hybrid fibre in a hybrid fabric is preferably above 0.05 GPa, such as above 0.1 GPa, preferable above 0.3 GPa, more preferable above 0.5 GPa, further preferable above 0.7 GPa, yet further preferable above 0.8 GPa, most preferable above 0.9 GPa.

In an embodiment, the first fibre is a polymer selected from the group of polymers listed above in respect of second fibre. Preferred is polymers of polyolefins e.g. LDPE (low density polyethylene), LLDPE (linear low density polyethylene), PET, PP (polypropylene), UHMWPE fibre, which is not oriented. This may for example be UHMWPE fibre which is not gelspun and having a melting point below the melting point of gel-spun UHMWPE.

The first type fibre is usually at least partially melted during processing into the medical component and/or into a final medical device and hence, the second type fibre may be transformed into a non-fiber phase (such as a continuous matrix) during processing. It is emphasized that the scope of the present invention as defined by the claims encompasses such embodiment of the hybrid fabric, medical components and medical devices. It is highly preferred that the second type fibre retain the initial fibre properties after processing, as the desired wear characteristic of the second fibre and hence of the hybrid fabric overall typically relates directly to the fibre properties of the second fibre.

The useful combinations of second and first fibre in the hybrid fabric may vary considerably. It was however found to be highly advantageous that the Young's modulus of said second fibre is at least 150% or even at least 200% of the Young's modulus of the first fibre, i.e. at least two times the Young's modulus of the first fibre. This allowed for a suitable difference in properties (including for example stiffness, strength, wear resistance and/or melting point) between the two sides of the hybrid fabric to realize improved overall wear properties of the medical component while realizing a strong bond between the fabric and the structural component of the medical component.

In a preferred embodiment, where the first fibre has a low Young's modulus, such as a modulus of 15 GPa or less, the Young's modulus of the second fibre is at least 500% or even at least 1000% of the Young's modulus of the first fibre. This allows for a highly processable system, which at the same time provides a highly durable (wear) surface of the final medical component or medical device.

In another embodiment, it was found to be advantageous that the Young's modulus of the first fibre is at most 150 GPa and the Young's modulus of the second fibre is at least 50 GPa.

In the particularly preferred embodiment where the second fibre is high crystalline UHMWPE, it is preferred that the Young's modulus of the first fibre is between about 5 GPa to about 50 GPa, such as between about 7 GPa to about 20 GPa, as this allows for a system with very good wear properties based on the high crystalline UHMWPE and good processability based on the first fibre, such as preferably HDPE or another polymer having a melting point below the melting point of high crystalline UHMWPE.

In the preferred embodiment, where the first fibre is high crystalline UHMWPE, it is preferred that the Young's modulus of the second fibre is between about 140 GPa to about 250 GPa, such as between about 150 GPa to about 200 GPa, as this allows for a system with very high shape stability. For such systems, it is preferred to include an adhesive (film) between the structural component and the hybrid fabric to enhance bonding between the structural component and the hybrid fabric. Furthermore, it is often advantageous to arrange the first fibre at the wear surface and the second fibre towards the structural component of the medical component, if the wear properties of the second fibre lead to unacceptable bearing properties of the medical component, i.e. too high wear of the natural or artificial component that the medical component will wear against during use.

The most suited first fibre is a polymeric fibre, which may retain the polymeric structure also in the preparation of the medical component. In one embodiment, a portion of the first fibre may however melt in the process of preparation the medical component. This embodiment has the advantage, that an additional matrix material is not needed for connecting the hybrid fabric to the structural component.

The first fibre may be semi crystalline fibre.

The first fibre may also be a matrix material of the material mentioned herein.

In another embodiment at least a part of the first fibre are hybrid fibre comprising first fibre filaments and second fibre filaments. Hybrid fibre may comprises 1-99% of second fibre filaments, such at less than 90%, e.g. less than 80%, such as less than 70%, e.g. less than 60%, such as less than 50%, e.g. less than 40%, such as less than 30%, e.g. less than 20%, such as less than 10%, e.g. less than 5%. In one embodiment, it is preferred that the first fibre are hybrid fibre comprising first fibre filaments and 1-30% of second fibre filaments. In another embodiment, it is preferred that the fabric comprises about 50% hybrid fibre, such as between 40% and 60%. A fibre may typically comprise 1-1200 fibre filaments and preferably between about 25 to 800 filaments, such as about 50 fibre filaments in the first fibre.

The second fibre of the hybrid fibre may be any second fibre specified elsewhere herein. Preferred is a second fibre selected from Polyamide and UHMWPE. More preferred is a second fibre of UHMWPE.

In an embodiment the second fibre comprises 0-100% of the hybrid fabric. The remaining amount of the second fibre may be at least one second fibre as specified elsewhere herein.

The first fibre may also comprise 0-100% of the hybrid fabric. The remaining amount of the first fibre may be at least one first fibre as specified elsewhere herein.

The amount of second fibre in the hybrid fabric may be 10-99%. The amount of first fibre in the hybrid fabric may be 1-90%.

In a preferred embodiment the amount of second and first fibre in the hybrid fabric is substantially equal i.e. each between 40-60%, such as each about 50%. The calculation is based on the weight of the fibre material. Thus the percentages given may also include the second fibre and first fibre of hybrid fibre used in the preparation of a hybrid fabric. The amount of second fibre preferable has majority in the second side of the hybrid fabric and the first fibre preferable has majority in the first side of the hybrid fabric i.e. at the side to be adhered to a structural component. In a highly preferred embodiment, the second side of the hybrid fabric is substantially free from first fibre, as it surprisingly found that even small amounts of first fibre present at the second side of the medical component significantly reduced the wear properties of the medical component during use.

The hybrid fabric may comprise at least two layers, one of the layers comprises the second fibre and the other of the layers comprises the first fibre. The layers of the hybrid fabric may be true layers sewed together, truly interwoven layers or may not be true layers but only used to describe the amount of the second fibre and the first fibre in an area (layer) of the hybrid fabric.

The hybrid fabric may comprise at least three layers, one of the layers comprises the second fibre and the other of the layers comprises the first fibre the third layer being the middle layer comprises substantially 50% (40-60%) of the second fibre and 50% (40-60%) of the first fibre.

The second fibre and the first fibre of the hybrid fabric may be interwoven is such a way that the layers have a transition zone such that one layer merge into the adjoining layer in respect of the amount of the soft and second fibre. In a highly preferred embodiment, the hybrid fabric comprises at least two different layers of which at least one layer is free from first fibres. The hybrid fabric may also comprise at least three different layers, such as four, five, six or even more layers. Hybrid fabrics with two or three layers are most preferred, as such fabrics allow for good separation of first fibers from the second side of the fabric while yet being affordable.

The hybrid fabric may be manufactured by different techniques; hereby it may be woven fabric, knitted fabric, crochet fabric, 3D fabric, such as a 3D weaving; spacer fabric, felted fabric, tufted fabric, (filament) winded fabric and/or non-woven fabric.

The hybrid fabric has a first side and a second side, the first side comprises first fibre and the second side of the hybrid fabric comprises nearly only second fibre. Surprisingly it was found that a fabric with highly crystalline fibre and semi crystalline fibre on the first side is doubling the adherence tension/strength of the fabric adhered to the polymeric component when compared to a fabric without the combination of highly crystalline fibre and semi crystalline fibre on the first side.

The hybrid fabric can be described as a void fabric before the hybrid fabric is adhered to a structural component. By void is meant that the fabric has an inner volume not occupied by any material except single fibers or fibre monofilaments that keeps the structural integrity of the fabric. The void volume of a fabric may be up to 99% of the total volume defined by the outermost fibre of the hybrid fabric. Preferred is about 75%.

When the hybrid fabric is adhered to a structural component the hybrid fabric is typically pressed together and a volume of the first fibre may be melted, or molten material is pressed into the void space, hereby the void volume of the hybrid fabric is decreased The void volume may be totally eliminated or leading to a 0.1 to 20 mm thick connected hybrid fabric with similar or different fabric structures on each side. In a preferred embodiment the hybrid fabric has substantially 100% second fibre at the outside of the medical component i.e. at the second side of the fabric, the wear surface, and lesser towards the first side of the fabric i.e. towards the structural component.

In another preferred embodiment the hybrid fabric has substantially 100% second fibre at the second side and substantially 100% first fibre at the first side. In a preferred embodiment the hybrid fabric is prepared from about 50% second fibre and 50% first fibre.

In an embodiment the hybrid fabric is produced from hybrid fibre made of about 50% (40-60%) second fibre and 50% (40-60%) first fibre.

The second type fibre usually is higher in wear resistance than the first type fibre. However, the wear resistance of the final medical component or medical device should be considered towards the (natural) component, such as a bone part, which is wearing against the medical component of the present invention. Hence, in one embodiment of the invention, the first fibre and the second fibre are exchanged leading to the fibre with relative high Young's modulus being arranged predominantly towards the structural component and the fibre with relatively low Young's modulus being arranged towards the wear surface. Such an embodiment typically involves fibres having very high Young's modulus and/or very high wear resistance during the actual use. Here, the connection between the hybrid fabric and the structural component should be considered carefully, and it was found to be particularly advantageous to include an adhesive (film) in the interface between the structural component and the hybrid fabric.

### Structural Component

The medical component comprises a structural component which by itself comprises at least one type of polymer ceramic, glass and/or metal. Preferably the structural component comprises a polymer selected from the group of polyolefinic polymers, polyethylene, polypropylene, polyacrylates, polystyrene, polytetrafluorethylene, polyvinylalcohol, polyethylene oxides, polyvinylpyrrolidon, polysilanes, polyurethanes, polyethers, polyamides, polyesters, polyalkyl acrylates, nylon, rubber and/or epoxy resins. Preferably the structural component comprises UHMWPE or PEEK. Most preferred are structural components of UHMWPE.

Another type of preferred structural components are hybrid fabrics as described above. In this embodiment, two hybrid fabrics are connected thereby forming an implant with two wear surfaces. Such a medical component could for example advantageously be used as an interpositional arthroplasty.

The polymer or polymeric material of the structural component may be selected from the group of the polymeric material described elsewhere herein as suitable for use as second fibre.

The structural component to be comprised in the medical component is preferably selected from the group of implant used in joints of knees, hip, shoulders, fingers, wrist, elbow, spine, neck, loin, toes and ankles.

The structural component may be produced by any known methods.

In a preferred embodiment, the structural component may be cup-shaped and comprise a polyolefin material. Such a structural component is particularly advantageous in that it is highly useful as a liner in many applications. The polyolefin material may be UHMWPE, such as amorphous UHMWPE.

In an embodiment the medical device may have a thickness of between 2 and 20 mm, such as a cup-shaped device with a thickness of 3-15 mm. This allows for both very thin liners as well as full thickness implants, such as artificial cartilage. In a further preferred embodiment the hybrid fabric is connected to the inner side of the cup-shaped device. By inner side is meant the side having the smallest diameter if drawing a circle or a sphere along the lines of the two sides of the cup-shaped device. This allows the hybrid fabric to enforce the area of the cup-shaped device, which is typically exposed to wear.

The structural component may for example be an injection moulded component or a compression moulded component. The component which is produced by injection moulding will have signs of the inlet(s) from the manufacturing process and signs of the surface of the casting mould which in many cases results in a smooth surface of the component, whereas the compression moulded and subsequent machined component will have lines or marks from the machining when the component is processed into the desired shape. The different manufacturing techniques can also be determined from the tension lines of the components.

The structural component can be a component, which is to be secured in the body, or which is to be attached to another component, which is to be secured in the body of an individual. The structural component to which the hybrid fabric is to be adhered to may be a liner, which can be connected to a metal backing or socket which will be secured to e.g. the pelvis of an individual.

If the structural component is made of metal, this component may be macroscopic or microscopic porous at least in the area where a hybrid fabric is to be adhered to the structural component. An adhesive may or may not be used when the hybrid fabric is connected to the structural component.

### Adhesive (Film)

The medical device described herein may also comprise a polymeric film or an adhesive or sizer located between the hybrid fabric and the structural component. Such an adhesive need not be visible in the final product as the adhesive may melt during manufacturing and be a part of the first side of the hybrid fabric in the final medical device. The type of material used as adhesive and/or the amount of first fibre in the final medical device may indicate the use of an adhesive in the production process of the medical device.

The polymeric film or adhesive or sizer may be selected from the group of polymeric materials described in respect of the second fibre or first fibre, however, the adhesive need not be a fibre, but may alternatively be at least partially an amorphous phase.

Preferably, the adhesive used in the production of a medical device if present is the same type of polymer as used as the soft polymer fibre in the hybrid fabric. Thus all polymers mentioned as soft polymers may also be used as adhesive materials.

The medical component may be produced by locating a polymeric film or adhesive or sizer between the hybrid fabric and the structural component before performing the step of connecting the hybrid fabric to the structural component.

The film can be of a thickness between 5 and 3000 μm, and preferably between 10 and 1000 μm.

In a preferred embodiment the polymeric film has a thickness of about 100-300 μm. Surprisingly it was found that the use of a film or an adhesive result in a doubling of the adherence tension/strength of the fabric to the polymeric component. Furthermore, a medical component produced from a hybrid fabric with second fibre and first fibre on the first side of the hybrid fabric i.e. on the side connected to a structural component together with a film between the hybrid fabric and the structural component result in a factor of about four of the adherence tension/strength of the hybrid fabric adhered to the polymeric component when compared to a fabric of only only second fibre on the first side and without using the film or adhesive.

### Production of the Medical Device

Another aspect of the invention is related to a method for the production of a medical component. The method comprises

i. providing a hybrid fabric, and
ii. providing a structural component,
iii. adhering the hybrid fabric to the structural component, and hereby
iv. obtaining a medical device or a component of a medical device.

The hybrid fabric and the structural component may be anyone described elsewhere herein and combined in any combination. The hybrid fabric comprises at least a second fibre and a first fibre as further described above. In the production of the medical device the hybrid fabric is connected to the structural component by connecting the first side of the hybrid fabric to the structural component by chemically bonding, such as adhering, and/or physically bonding, such as having intermingled elements and/or having mechanically locking members. Physically bonding via at least partially melting of the hybrid fabric, particularly at least partially melting of the first fibre, is preferred, as this may be conducted without introducing another material into the medical component.

The hybrid fabric may be preformed into a desired shape before performing step iii for example by compression moulding. The step iii of connecting the hybrid fabric to the structural component may be performed at a temperature above the melting point of the first fibre, which allows for a more intimite connection between the fabric and the structural component.

Step iii of connecting the hybrid fabric to the structural component may also be performed by compression moulding, which allows for a highly reproducible results with very low degree of deformation after completion of the moulding process.

In another embodiment, the structural component is made of a polymeric material suitable for injection moulding, and the structural component may be produced by injection moulding the polymeric material onto the first side i.e. to the anchoring layer of the hybrid fabric. The hybrid fabric may be pre-formed before performing this injection moulding. The polymeric material suitable for injection moulding may have a temperature of 100-300° C. just before the polymeric material enters the mould for moulding the polymeric material.

Step iii may be performed in a mould, and the mould may be pre-heated or having a means for stabilizing the temperature to a temperature of between 50 and 100° C. before performing step iii.

In the production of a medical device a polymeric film may be located between the hybrid fabric and the structural component before performing step iii of connecting the hybrid fabric to the structural component.

In another embodiment, the hybrid fabric is provided by (filament) winding of second and first fibre. The winding may be conducted directly onto the structural component, which is preferred due to the relatively simple manufacturing process, or the winding may be conducted for example on a separate mantel followed by at least partially fixation of the winded fibres (for example by heat optionally including shaping or addition of an adhesive) before connecting the hybrid fabric to the structural component. Winding not directly on the structural member allows for application of a winded fabric also on the inner surface of cup shaped structural member.

Furthermore it was found that the hybrid fabric and/or the structural component advantageously may be pretreated before performing step iii to enhance the connection between the hybrid fabric and the structural component in the final medical component. Suitable pretreatment was found to be one of more of the pretreatments selected from the group of plasma treatment, sizing, coating with a soft polymer, calendaring (for example to reduce thickness or un-evenness), mechanically and/or chemical grinding or polishing, etching, and/or grafting. Especially the first side of the hybrid fabric can be subjected to a plasma-treatment such as an oxidation and/or a coating with soft polymers before connecting to the structural component. Surprisingly, the plasma-treatment need not to be performed right before performing step iii, but may be performed in advance.

### Use

The medical component of the invention is highly suitable as part of a medical device or in some cases a medical device; such as an implant; may consist solely of the medical component according to the invention.

The hybrid fabric as described herein above may be used for at least partially covering a medical device. This may be as a cover performed in an area subjected to tear and/or wear when the medical implant is located in an individual. The coating may be performed on a medical implant to be used in a joint of an individual.

The medical component described herein may be used such that the second side of the hybrid fabric is to be aligned to a natural component; such as a bone or a bone part; or non-natural component; such as an implant; of an individual. The medical device described herein may be used such that the natural or non-natural component rub towards the hybrid fabric.

A highly advantageous application of the medical component described herein is the use as a liner to be used in an acetabular component. The very high wear resistance of particularly medical components having highly crystalline UHMWPE as the second fibre allows for the preparation of very thin liners with excellent wear resistance, hence allowing for reducing or postponing the need for a full hip replacement.

The medical component described herein may be used such as an acetabular component. When the hybrid fabric is used in the manufacture of a liner for an acetabular component or used for an acetabular component, a natural or non-natural femoral head e.g. a metal or ceramic ball is to be located next to the hybrid fabric adhered to the liner or acetabular component.

The medical component described herein may be used in an implant for total hip alloplasty, total hip replacement, total knee alloplasty or total knee replacement due to the very improved wear resistance realized by the addition of the hybrid fabric.

A medical component produced according to the description herein may be capable of being formed to suit into parts of the organism as described elsewhere herein. Especially the medical component is suitable to be used in animals, such as mammals and human beings, preferred is human beings. The animals, to which the medical device may be utilised, may be selected from the group of mammals, such as but not limited to horses, dogs, cats, cows and monkeys.

In one embodiment the medical component is especially constructed to be utilised to support, hold, sustain, bear, carry, replace or displace any constitution within the individual e.g. the mammalian body, which comprises high shape stability and good wear resistance. The product can be adapted not to interfere with intra-articular or other components when the medical component is in the body of a human. The product as medical device may be but is not limited to be used as implants or as components in implant to be used in joints of knees, hip, shoulders, fingers, wrist, elbow, spine, neck, loin, toes and ankles. Especially the devices are used in diseased patients with osteoarthritic degeneration of joints.

The medical component as described herein may be produced in a number of sizes corresponding to the natural variety of the bones within the joint where it is intended to be used as well as to the differences in bone size due to the age or size of individuals. The hybrid fabric as described herein may be used as a coating or cover of any medical device. Especially the hybrid fabric is suited to coat medical implants that are to be in connection with an individual, also the hybrid fabric is beneficial when used to coat or cover at least a part of a medical device, which is to be used as an implant. The hybrid fabric is especially qualified for areas of an implant, which is subjected to wear and/or tear such as the movement performed in a joint where the movement of the individual makes the ends of bones to rub towards each other. Surprisingly if was found that the second fibre of the hybrid fabric, which is the outermost surface of the implant and being exposed to wear, significantly reduces the number of particles released in the individual. Furthermore and even more surprisingly, it was found that the particles actually released are larger than when implants is used without a surface as obtained by the second fibre of the hybrid fabric.

A prosthetic device as defined herein may be used for improving the wear in a joint of a vertebrate such as a human, by inserting into the joint said prosthetic device. An embodiment relates to a method for establishing slidability and/or pressure distribution in a joint of a vertebrate such as a human, comprising inserting into the joint, a prosthetic device, preferably a prosthetic device as described elsewhere herein.

### DETAILED DESCRIPTION OF THE DRAWINGS

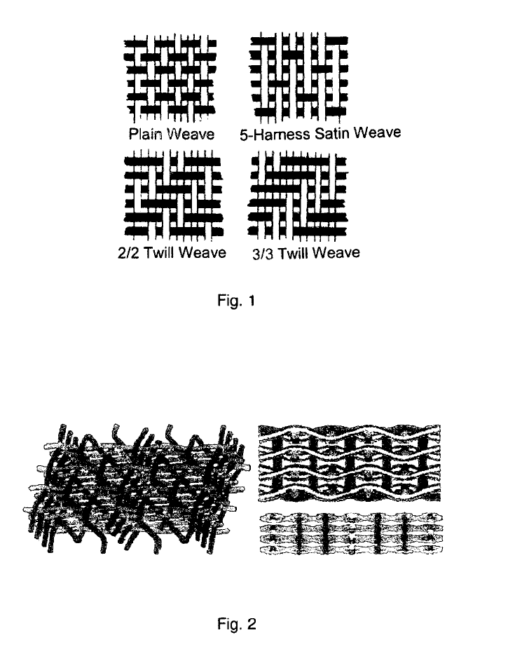

FIG. 1 shows various typical 2D weave structures, which may be used in respect of the preparation of a 3D hybrid fabric as described herein. Similar or different layers of the 2D weave may be use to prepare a hybrid fabric, also the different weaving techniques may be used in the preparation of a 3D hybrid fabric. The shown examples are plain weave, 5-harness satin weave, 2/2 twill weave and 3/3 twill weave.

FIG. 2 shows a 3D weave structure with multi-layer interlaced. The figure illustrates the orientation of different fibre in the hybrid fabric, where some fibres are running in only one direction, whereas other fibre are running in more than one direction.

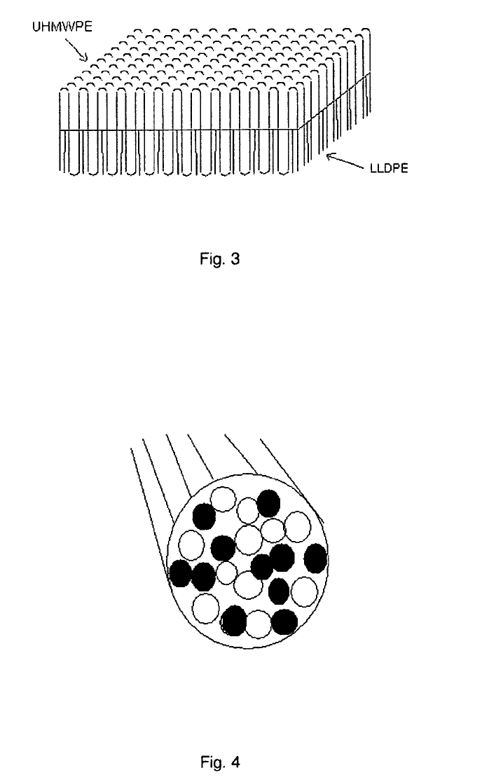

FIG. 3 illustrates a hybrid fabric (composite fabric) made of UHMWPE as the top layer (wear layer=second side) and UHMWPE and LLDPE in the ratio 1:1 in the lower layer (anchoring layer=first side). The production of the hybrid fabric need not be by providing at least two layers of the different fiber, but rather by weaving the different fibre directly into the illustrated structure. The thickness of the hybrid fabric may for example be 1.5-25 mm, which can be compressed to for example 0.7 mm when the medical device is produced.

FIG. 4 illustrates an example of the fibre filaments of a hybrid fibre by a cross section of the hybrid yarn. In this example 50% of the fibre filaments are first fibre indicated by dark circles and 50% are strong fibre filaments indicated by open circles. The hybrid fibre may have from 1% to 99% second fibre, the remaining fibre being first fibre. The number of fibre filaments may be up to 1000, normally 25-125 fibre filaments are used. The figure illustrates a hybrid fibre with 22 fibre filaments. A hybrid fibre within the inventive idea may also be obtained by mixing of second fibre and first fibres, for example by twisting or braiding. Hybrid fibres may also comprise a mixture of first fibre with another type of first fibre or a mixture of second fibre with another type of second fibre.

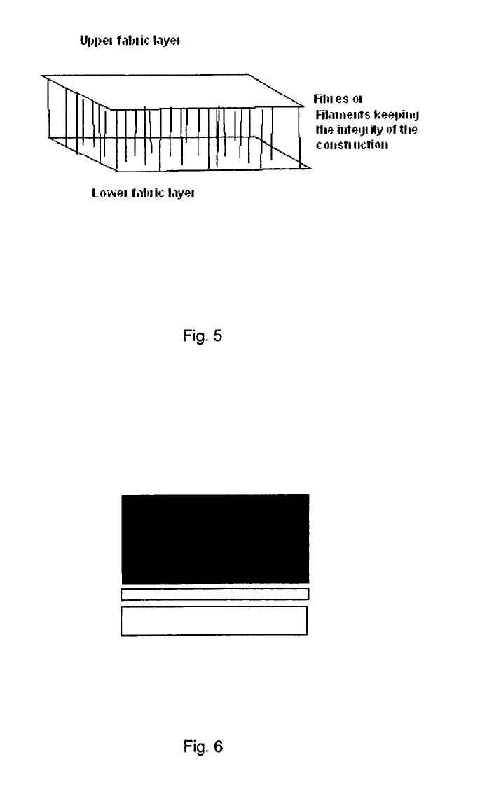

FIG. 5 illustrates a spacer fabric as a hybrid or composite fabric. Between an upper fabric layer and a lower fabric layer, fibres of filaments are keeping the integrity of the construction.

FIG. 6 illustrates the structure of a medical device. The upper part is the structural component, e.g. a UHMWPE backing of e.g. 7 mm thickness. The middle part is a soft matrix layer (adhesive layer) which is for example 100 μm thick. The lowest layer is a hybrid fabric e.g. a 3D hybrid fabric of e.g. 500 μm thickness. Some of the layers need not be visible in the produced medical component. For example, the matrix material (matrix layer) may be integrated into the hybrid fabric to an extent where it may not be distinctable from the hybrid fabric anymore. The structural component as well as the materials for the hybrid fabric and the adhesive layer may be anyone described elsewhere herein.

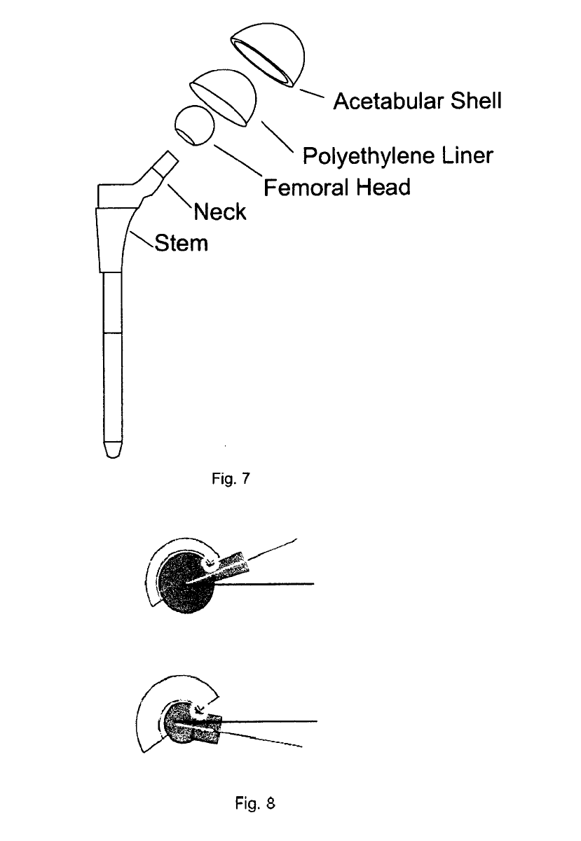

FIG. 7 illustrates an implant for total hip replacement with an acetabular shell, a liner, a femoral head, a neck and a stem. The liner may be covered with a hybrid fabric at the side to be in connection with the femoral head (ball) to improve the wear resistance of the liner.

FIG. 8 illustrates two examples of a femoral head located in a liner or acetabular shell. In the upper example a thin liner/acetabular shell makes it possible to use a larger head of the femoral head, flexion above 90° is possible. In the lower example a thick liner/acetabular shell makes it only possible to use a small head of the femoral head only flexion below 90° is possible. The outer size of the cups is equal. A hybrid fabric is used in the liner/acetabular shell of the upper example, where the wearability of the hybrid fabric makes it possible to decrease the thickness of the liner/acetabular shell considerably.

### Tensile Property Measurement:

Tensile tests were carried out on an Ingstron Z010 tensile tester equipped with a 1 kN load cell and Instron parabolic fiber grips, in accordance with ASTM D885M, using a nominal gauge length of the fibre of 500 mm. Tensile strength was determined from the force at break and the linear density measured on each individual sample. Tensile modulus was determined as the chord modulus between 0.3 and 1.0% strain.

### EXAMPLES

### Example 1

### Preparation of a Hybrid Fabric

Hybrid fabric was constructed using a 3D weaving technique. The second surface the fabric contained only UHMWPE fibre (Dyneema® Purity®). The first surface consisted of 50 wt-% UHMWPE fibre and 50 wt-% LLDPE fibres spun with 2 wt-% nylon to facilitate weaving. This was it was possible to compression mould performs with two different surfaces, namely one surface with un-molten UHMWPE fibre and one with molten LLDPE in an un-molten UHMWPE fibre matrix for adhering to a structural component.

### Example 2

### Production of a Liner for an Acetabular Component

A hybrid fabric prepared as in Example 1 was preformed into a cup-shaped design by compression moulding. The inner side of the cup had the second fibre at the surface and the outer side of the cup had the first fibre at the surface.

Amorphous UHMWPE was injection moulded directly onto the surface of the preformed hybrid fabric, such that a structural component was produced on the LDPE. The structural component also had a cup-shaped design.

The cup-shaped medical component can be used as an implant for a liner in an acetabular component for a partially or totally hip replacement.

### Example 3

### Peel Strength

UHMWPE-fibre coated implants were prepared and the adhesion between fibre and matrix was investigated. A hybrid fabric as prepared in Example 1 was connected to a structural component of UHMWPE granulate material ATOFINA Gur by injection moulding the structural component (UHMWPE granulate) directly on a perform onto the LLDPE side mentioned in example 2.

As a comparison, the example was repeated using a fabric of 100% hard fibre, with the same structural component using the same manufacturing technique (injection moulding of the UHMWPE granulate directly onto the LLDPE).

The Peel strength was measured by peeling of the UHMWPE fabric from the structural component, severing the LLDPE adhesion layer in between. The test quantitatively measured the force necessary to peel off the fabric and subsequent calculated the work used in the process. The results are provided in Table 1, where each data point represents an average of 8 samples measured.

### Example 4

### Peel Strength

Example 3 was repeated with the additional step of applying a LLDPE film layer (thickness 100 micrometers) onto the hybrid fabric and performing the LLDPE film layer and the hybrid fabric by compression moulding-shaping technique. Then the structural component is injection moulded directly onto the LLDPE film on the fabric as describe in Example 3. Thereafter the peal strength was measured by the same technique as in Example 3. The results are provided in Table 1, where each data point represents an average of 8 samples measured.

TABLE 1

| | | Tool | | | |
|---|---|---|---|---|---|
| | Preparation | Temperature at | | | Peel |
| Test | Conditions of | injection | | LDPE | Work |
| Name | preform | moulding | Fabric Type | layer | (J/m²) |
| C20 | Heat treatment | 80° C. | Pure | No | 2120 |
| | 135° C. 1 min, | | UHMWPE | | |
| | | | (Dyneema) | | |
| C50 | Heat treatment | 50° C. | Hybrid | No | 4742 |
| | 135° C. 1 min, | | | | |
| C80 | Heat treatment | 80° C. | Hybrid | No | 4920 |
| | 135° C. 1 min, | | | | |
| D50 | Heat treatment | 50° C. | Hybrid | Yes | 10708 |
| | 135° 1 min | | | | |
| D80 | Heat treatment | 80° C. | Hybrid | Yes | 11551 |
| | 135° 1 min | | | | |
| E80 | Heat treatment | 80° C. | Dynema | Yes | 5453 |
| | 135° 1 min | | Purity Klæde | | |
| F80 | Heat treatment | 80° C. | Hybrid | Yes | 11713 |
| | 135° 1 min | | | | |

Surprisingly it was found that using a hybrid fabric consisting of 75% UHMWPE-fibre and 25% LLDPE fibre resulted in an increase by a factor of at least two in peel strength of the connection between fabric and structural component, as compared to the peel strength realized between a fabric of 100% UHMWPE-fibre and the UHMWPE structural component.

Even more surprisingly, the very positive effect of the hybrid fabric could be even further enhanced to a total factor of at least four when combined with a LLDPE film layer. The effective adhesion increases the lamination strength thus considerably decreasing the risk of delamination in the implant liner during use.

### Example 5

### Wear Measurement

A hip implant liner using UHMWPE-fibres at the wear surface is designed and constructed as in example 3 above. The samples is subsequently mechanically tested in an ISO 14242, 6 stage hip simulator, simulating in vivo walking with a following ISO 17853 wear materials characterization.

From wear data it is concluded that the number of particles is significantly reduced using UHMWPE fibres at the wear surface. Tests have shown that wearing on UHMWPE fabric adhered to a structural component typically yield as low as 10⁸ particles per million cycles. This should be compared with typical prior art values which indicates that in the order of 10¹¹ to 10¹² particles per million cycles are generated when wearing directly on amourphous UHMWPE as used in traditional implants.

It is emphasized that wear properties of different materials are highly depending on the actual application. The results of the standardized tests may therefore vary considerably from the material performance of real implants during use.

### Example 6

### Hybrid Material with Metal

A hybrid material with a metallic fibre, such as steel or tantalum, is interwoven with UHMWPE fibers. This hybrid material may be pressed into any desired shape, such as a cup shape of a sheet shape. The hybrid material is particularly useful as a hip implant liner, or an interpositional arthroplasty. Furthermore, the hybrid material may be utilized in covering (a part of) an implant for improving the wear resistance of the implant.

### Example 7

### Interpositional Arthroplasty

An interpositional arthroplasty is designed and constructed using a hybrid material as describe in example 6. A custom made in vivo like testing rig was designed using a combined tension/torsion servo hydraulic INSTRON 8874 test machine combining, biaxial movement, torsion movement and axial loading between suitable ceramic components mimicking the surface hardness of arthritic bone. The interpositional arthroplasty is tested in the rig to evaluate for deformation and wear. The implant shows no visible deformation or wear from the test rig, but still allows for possibly good functionality due to a high degree of material and final shape flexibility.

### Example 8

### Hybrid Material with Ceramic Fibre

Fibre of a ceramic material like ZrO₂ or Al₂O₃ and a soft fibre like LDPE are interwoven as describe above. The wear surface would then consist of a pure ZrO₂ or Al₂O₃ layer, which may easily and with a strong bond be adhered to the structural component by melting of the LPDE component.

### Example 9

### Effect of Pre Treating

Coating of the surface with hydrophilic polymer by plasma polymerization. A plasma reactor was custom made and had an electrode configuration that allowed for a uniform coating with a layer of polyvinyl pyrrolidone (PVP) on both the inside and the outside of an interpositional implant with a cup shaped configuration. The implant consisted of a hybrid fabric as described in example 1. The coating was subsequently physico-chemically investigated using TOFF-SIMMS, IR, XPS and a custom made mechanical test rig. The measurements confirmed that the layer of PVP was chemically bonded to the UHMWPE-fibres at the second side and protruding LDPE the matrix material at the first side. After the treatment it was possible to clean the samples for monomers etc. by using supercritical CO2.

The advantage of adding a PVP-layer to the implant is that the surface becomes hydrophilic, absorbing water and thus lowering the coefficient of friction and the wear rates, especially in the initial wear phases at the second side and at the same time improving the biocompatibility of the first side.

1. A biocompatible medical component comprising
a hybrid fabric comprising
i. at least one type of second fibre, and
ii. at least one type of first fibre, and
a structural component,
wherein the Young's modulus of said second fibre is at least 125% of the Young's modulus of the first fiber.

2. The medical component according to claim 1, wherein said structural component and said hybrid fabric are connected to each other such that a first side of said fabric is connected to said structural component, preferably said fabric is chemically bonded to said structural component, such as adhered, and/or physically bonded, such as having intermingled elements and/or having mechanically locking members.

3. The medical component according to claim 1, wherein the Young's modulus of said second fibre is at least 150% of the Young's modulus of the first fiber, preferably the Young's modulus of said second fibre is at least 200% of the Young's modulus of the first fiber, more preferably the Young's modulus of said second fibre is at least 500% of the Young's modulus of the first fibre, such as at least 1000% of the Young's modulus of the first fibre.

4. The medical component according to claim 1, wherein the Young's modulus of the first fibre is at most 150 GPa and the Young's modulus of the second fibre is at least 50 GPa.

5. The medical component according to claim 1, wherein said second fibre is
a metal fibre selected from the group of metals normally used for implants to be located in an individual; like stainless steel, tantalum, titanium or cobalt/chrome steels,
a polymer fibre selected from the group of polyolefins e.g. UHMWPE; aramid or para-aramid (Kevlar), polyketones e.g. PEEK (polyetheretherketone), polyamide e.g. Nylon, polyester, polyvinylchloride and polyacrylonitrile and/or
a carbon fibre or carbon nano-tube fibre and/or
a ceramic or glass fibre selected from the group of SiC, Si₃N₄, Ca₃(PO₄)₂, SiO₂, CaO, MgO, K₂O, Na₂O, Al₂O₃, silicates, boro silicates, alumino silicates, BC, mineral fibers and bone ingrowth materials, such as PMMA, FiberLive™, tricalciumphosphate, hydroxyapatite covered materials, porous materials, such as tantalum, titanium or steel and/or
a natural fibre selected from the group of silk, spider silk, plant fibre e.g. cellulose and/or
fibre selected from the group of coated fibre, coextruded fibre and hybrid fibres and/or
any mixture thereof.

6. The medical component according to claim 1, wherein said second fibre is high crystalline fibre.

7. The medical component according to claim 1, wherein said first fibre is a polymer selected from the group of polyolefins e.g. LLDPE, LDPE, PET, PP, UHMWPE fibre and a mixture of these which polyolefin preferably is not oriented.

8. The medical component according to claim 1, wherein said first fibre is semi crystalline fibre.

9. The medical component according to claim 1, wherein at least a part of said first fibre are hybrid fibre comprising first fibre filaments and second fibre filaments, preferably said hybrid fibre comprises 1-30% of second fibre filaments.

10. The medical component according to claim 1, wherein said second fibre of said hybrid fibre is selected from polyamide, such as Nylon.

11. The medical component according to claim 1, wherein said second fibre comprises 0-100% of said hybrid fabric.

12. The medical component according to claim 1, wherein said first fibre comprises 0-100% of said hybrid fabric.

13. The medical component according to claim 1, wherein the amount of second and first fibre in said hybrid fabric is substantially equal.

14. The medical component according to claim 1, wherein said hybrid fabric substantially comprises at least two layers, one of said layers comprises said second fibre and the other of said layers comprises said first fibre.

15. The medical component according to claim 1, wherein the hybrid fabric comprising at least two different layers of which at least one layer is free from first fibres, preferably the hybrid fabric comprising at least three different layers.

16. The medical component according to claim 1, wherein said hybrid fabric substantially comprises at least three layers, one of said layers comprises said second fibre and the other of said layers comprises said first fibre the third layer being the middle layer comprises substantially 50% of said second fibre and 50% of said first fibre.

17. The medical component according to claim 1, wherein said second fibre and said first fibre of said hybrid fabric are interwoven is such a way that the layers have a transition zone such that one layer merge into the adjoining layer in respect of the amount of the soft and second fibre.

18. The medical component according to claim 1, wherein said hybrid fabric is selected from the group of woven fabric, knitted fabric, crochet fabric, embroided fabric, 3D fabric, felted fabric, tufted fabric, (filament) winded fabric and/or non-woven fabric.

19. The medical component according to claim 1, wherein said structural component comprises at least one type of polymer, ceramic, glass, metal, and/or a hybrid fabric.

20. The medical component according to claim 1, wherein said structural component is selected from the group of implant used in joints of knees, hip, shoulders, fingers, wrist, elbow, spine, neck, loin, toes and ankles.

21. The medical component according to claim 19, wherein said polymer of said structural component is selected from the group of polyolefinic polymers, polyethylene, polypropylene, polyacrylates, polystyrene, polytetrafluorethylene, polyvinylalcohol, polyethylene oxides, polyvinylpyrrolidon, polysilanes, polyurethanes, polyethers, polyamides, polyesters, polyalkyl acrylates, nylon, rubber and/or epoxy resins.

22. The medical component according to claim 1, wherein said structural component is cup shaped and comprises a polyolefin material.

23. The medical component according to claim 22, wherein said polyolefin material is UHMWPE, preferably said UHMWPE is amorphous UHMWPE.

24. The medical component according to claim 22, wherein said cup shaped component has a thickness of 3-15 mm.

25. The medical component according to claim 1, wherein said hybrid fabric is connected to an inner side of said cup.

26. The medical component according to claim 1, further comprising a polymeric film between said hybrid fabric and said structural component.

27. The medical component according to claim 1, wherein said polymeric film is selected from the group of polyolefinic polymers, polyethylene, polypropylene, polyacrylates, polystyrene, polytetrafluorethylene, polyvinylalcohol, polyethylene oxides, polyvinylpyrrolidon, polysilanes, polyurethanes, polyethers, polyamides, polyesters, polyalkyl acrylates, nylon, rubber and/or epoxy resins.

28. The medical component according to claim 1, wherein said structural component is an injection moulded component or a compression moulded component.

29. A method for the production of a medical component, said method comprising
i. providing a hybrid fabric, and
ii. providing a structural component,
iii. connecting said hybrid fabric to said structural component, and hereby
iv. obtaining a medical component.

30. The method according to claim 29, wherein said hybrid fabric comprises at least one type of second fibre and at least one type of first fibre.

31. The method according to claim 29, wherein said hybrid fabric is connected to said structural component by connecting the first side of said hybrid fabric to said structural component by chemically bonding, such as adhering, and/or physically bonding, such as having intermingled elements and/or having mechanically locking members.

32. The method according to claim 29, wherein said hybrid fabric is preformed into a desired shape before performing step iii.

33. The method according to claim 29, wherein said hybrid fabric is preformed into a desired shape by compression moulding.

34. The method according to claim 29, wherein step iii of connecting said hybrid fabric to said structural component is performed at a temperature above the melting point of said first fibre, preferably said temperature is below the melting point of said second fibre.

35. The method according to claim 29, wherein step iii of adhering said hybrid fabric to said structural component is performed by compression moulding.

36. The method according to claim 29, wherein step i is conducted after step ii and the hybrid fabric is provided by (filament) winding of second and first fibre preferably directly onto said structural component.

37. The method according to claim 29, wherein said structural component is made of a polymeric material suitable for injection moulding, and said structural component is produced by injection moulding said polymeric material onto the first side of said hybrid fabric.

38. The method of claim 37, wherein said polymeric material suitable for injection moulding has a temperature of 100-300° C. just before the polymeric material enters the mould for moulding the polymeric material.

39. The method according to claim 29, wherein step iii is performed in a mould, and said mould is pre-heated to a temperature of between 50 and 100° C. before performing said step iii.

40. The method according to claim 29, wherein a polymeric film is located between said hybrid fabric and said structural component before performing step iii of connecting said hybrid fabric to said structural component.

41. The method according to claim 29, wherein said hybrid fabric is further subjected to a pretreatment before performing step iii, preferably the pretreatment is selected from the group of plasma treatment, sizing, calendaring, (mechanically) grinding or polishing, etching, and/or grafting.

42. A medical device comprising the medical component according to claim 1, preferably said medical device consisting of said medical component.

43. Use of the medical component according to claim 1 in a medical device, such as an implant.

44. The use of a medical component as defined in claim 1 for improving the wear in a joint of a vertebrate such as a human, by inserting into the joint said prosthetic device.

45. A method for establishing slidability and/or pressure distribution in a joint of a vertebrate such as a human, comprising inserting into the joint, a medical device of claim 42.

46. Use of a hybrid fabric comprising a hybrid fabric comprising
a. at least one type of second fibre, and
b. at least one type of first fibre,
as specified in claim 1
for at least partially covering a medical device.

47. The use according to claim 46, wherein an area subjected to tear and/or wear during use is at least partially covered by the hybrid fabric.

48. The use according to claim 46, wherein said medical device is used in a mammal joint.

49. Use of a medical component according to claim 1, wherein a second side of said hybrid fabric is to be aligned to a natural component; such as a bone or bone part; or non-natural component;
such as an implant; of an individual.

50. Use according to claim 49, wherein said natural or non-natural component rubs toward said hybrid fabric.

51. Use of a medical device according to claim 49 as a liner to be used in an acetabular component.

52. Use of a medical device according to claim 49 as an acetabular component.

53. Use of a medical device according to claim 29 in an implant for total hip alloplasty, total hip replacement, total knee alloplasty or total knee replacement.
